(19) United States
(12) Patent Application Publication
(10) Pub. No.: US 2025/0249209 A1
(43) Pub. Date: Aug. 7, 2025

(54) VASCULAR INTERVENTION DEVICE AND DRUM ASSEMBLY FOR VASCULAR INTERVENTION DEVICE

(71) Applicant: PERAZAH INC., Ansan-si, Gyeonggi-do (KR)

(72) Inventors: Se Min OH, Jeju-do (KR); Hwan Taek RYU, Gyeonggi-do (KR); Jong Tae SEO, Gyeonggi-do (KR)

(73) Assignee: PERAZAH INC., Ansan-si, Gyeonggi-do (KR)

(21) Appl. No.: 18/841,009

(22) PCT Filed: Feb. 23, 2023
(86) PCT No.: PCT/KR2023/002615

§ 371 Date: Apr. 11, 2025

(30) Foreign Application Priority Data
Feb. 23, 2022 (KR) 10-2022-0023589

(51) Int. Cl. A61M 25/01

(52) U.S. Cl. CPC A61M 25/0113; A61M 2205/0216; A61M 2210/12

(57) ABSTRACT

There is provided a technique that includes: a supporter; and a drum assembly coupled to the supporter to be rotatable about a first axis, the drum assembly being configured to accommodate a flexible wire-type or tube-type surgical tool configured to be insertable into a blood vessel, and to allow the surgical tool to enter and exit through an entrance aligned on the first axis, wherein the drum assembly includes: a rotation part configured to be rotatable about a second axis and configured such that the surgical tool is wound around the rotation part in a circumferential direction about the second axis; and a pair of rollers configured to interlock with the rotation part and having a rotation axis parallel to the second axis, and wherein the drum assembly may be configured such that, when the rotation part rotates about the second axis, the surgical tool is guided to be pulled out from or inserted into the drum assembly through the entrance, and such that a portion of the surgical tool extending from the rotation part to the entrance is sandwiched between the pair of rollers.

### TECHNICAL FIELD

The present disclosure relates to a vascular intervention device. In detail, the present disclosure relates to a vascular intervention device that controls a flexible wire-type or tube-type surgical tool that is insertable into a blood vessel.

### BACKGROUND

Vascular interventions refer to minimally invasive treatments aimed at vascular diseases or cancer, and are mainly performed by inserting a thin catheter, with a diameter of several millimeters or less, percutaneously through a blood vessel to the lesion site under X-ray fluoroscopy, thereby reaching the target organ. Currently, the representative treatments of vascular interventions being performed in Korea and worldwide include, for example, trans-arterial chemoembolization (TACE) for liver cancer, percutaneous angioplasty, and endovascular stent grafting for aortic diseases.

Blood vessels are mostly divided into multiple branches or formed in a curved shape. Therefore, in order to prevent damage to blood vessels, vascular interventions use overlapping surgical tools with multiple stages of diameters, called a co-axial system of catheters and guide wires. At this time, because the blood vessels have branching points where a blood vessel is divided into several branches or curved sections, the operator must manually steer the catheters and guide wires precisely according to the direction of the blood vessels for insertion.

Conventional devices for inserting, extracting, or steering surgical tools have complex structures, making it inconvenient to replace surgical tools after a single use or to clean and reuse contaminated equipment.

### SUMMARY

The present disclosure provides a vascular intervention device that effectively implements the translational movement of a surgical tool such as a catheter and a guide wire.

In addition, the present disclosure provides a vascular intervention device that is capable of accurately controlling the translational movement of a surgical tool.

An aspect of the present disclosure provides a vascular intervention device. In an representative embodiment, the vascular intervention device may include: a supporter; and a drum assembly coupled to the supporter to be rotatable about a first axis, the drum assembly being configured to accommodate a flexible wire-type or tube-type surgical tool configured to be insertable into a blood vessel, and to allow the surgical tool to enter and exit through an entrance aligned on the first axis.

In an embodiment, the drum assembly may include: a rotation part configured to be rotatable about a second axis and configured such that the surgical tool is wound around the rotation part in a circumferential direction about the second axis; and a pair of rollers configured to interlock with the rotation part and having a rotation axis parallel to the second axis.

In an embodiment, the drum assembly may be configured such that, when the rotation part rotates about the second axis, the surgical tool is guided to be pulled out from or inserted into the drum assembly through the entrance, and such that a portion of the surgical tool extending from the rotation part to the entrance is sandwiched between the pair of rollers.

In an embodiment, the drum assembly may further include an interlocking mechanism configured to rotate the rotation part and the pair of rollers in conjunction with each other.

In an embodiment, the rotation part may include an outer peripheral surface, and the surgical tool is wound around the outer peripheral surface. The pair of rollers each may include an outer peripheral surface configured to come into contact with the surgical tool. The interlocking mechanism may be configured such that, when the rotation part and the pair of rollers rotate, the outer peripheral surface of the rotation part and the outer peripheral surfaces of the pair of rollers have a same outer peripheral linear velocity.

In an embodiment, the interlocking mechanism may be configured to rotate the pair of rollers in opposite directions from each other.

In an embodiment, the interlocking mechanism may include a plurality of gears that rotate in conjunction with each other. The plurality of gears may include: a rotation part gear coupled to the rotation part; a first driven gear coupled to one of the pair of rollers; and a second driven gear coupled to the other one of the pair of rollers and engaged with the first driven gear.

In an embodiment, the plurality of gears may include: a first intermediate gear engaged with the rotation part gear; and a second intermediate gear fixedly coupled to the first intermediate gear and having a pitch circle concentric with a pitch circle of the first intermediate gear. The first driven gear may be engaged with the second intermediate gear.

In an embodiment, the vascular intervention device may further include: a translation driver configured to rotate the rotation part and the pair of rollers. The translation driver includes: a translation driven shaft installed to the drum assembly and configured to rotate in mechanical conjunction with the rotation part; and a translation driving shaft installed to the supporter and configured to transmit power to the translation driven shaft.

In an embodiment, one of the translation driven shaft and the translation driving shaft may include a translation protrusion that axially protrudes, and the other one of the translation driven shaft and the translation driving shaft includes a translation groove configured to be engaged with the translation protrusion.

In an embodiment, the translation protrusion extends in a first direction perpendicular to a rotation axis of the translation driven shaft.

In an embodiment, one end of the translation protrusion in the first direction may be provided in a shape in which a circumferential width thereof increases in a radial direction.

In an embodiment, the vascular intervention device may further include: a rotation driver configured to rotate the drum assembly about the first axis with respect to the supporter. The rotation driver may include: a rotation driven shaft fixedly coupled to the drum assembly; and a rotation driving shaft installed to the supporter and configured to transmit power to the rotation driven shaft.

In an embodiment, the rotation driven shaft may include a hollow configured to accommodate the translation driven shaft. The rotation driving shaft may include a hollow configured to accommodate the translation driving shaft.

In an embodiment, one of the rotation driven shaft and the rotation driving shaft may include a rotation protrusion that axially protrudes, and the other one of the rotation driven shaft and the rotation driving shaft includes a rotation groove configured to be engaged with the rotation protrusion.

In an embodiment, the rotation protrusion may be provided in a shape in which a circumferential width thereof increases in a radial direction.

In an embodiment, the translation groove and the rotation groove may be configured to form an alignment groove by being mutually aligned. The translation protrusion and the rotation protrusion may be configured to form an alignment protrusion by being mutually aligned. The alignment protrusion and the alignment groove may be configured to be engaged with each other.

In an embodiment, the alignment protrusion and the alignment groove may be configured such that the alignment protrusion is inserted into the alignment groove in a direction perpendicular to the first axis.

In an embodiment, the drum assembly includes at least one guide roller configured to be rotatable about a rotation axis parallel to the second axis, arranged in a circumferential direction of the rotation part, and configured to come into contact the surgical tool wound around the rotation part.

In an embodiment, the vascular intervention device may further include: a surgical tool guide configured to accommodate a portion of the surgical tool extending from the rotation part to a space between the pair of rollers and guide movement of the surgical tool.

In an embodiment, the drum assembly may include an annular groove that is depressed in a direction of the first axis and extends in a circumferential direction around the first axis. The supporter includes a pin member that protrudes in the direction of the first axis and is inserted into the annular groove.

Another aspect of the present disclosure provides a drum assembly for a vascular intervention device. In an representative embodiment, the drum assembly may include: a drum housing configured to accommodate a flexible wire-type or tube-type surgical tool that is insertable into a blood vessel; an entrance aligned on a first axis to allow the surgical tool to enter and exit; a rotation part configured to be rotatable about a second axis with respect to the drum housing and configured such that the surgical tool is wound around the rotation part in a circumferential direction about the second axis; and a pair of rollers configured to interlock with the rotation part and having a rotation axis parallel to the second axis.

In an embodiment, the drum assembly may be configured such that, when the rotation part rotates about the second axis, the surgical tool is guided to be pulled out from or inserted into the drum assembly through the entrance, and such that a portion of the surgical tool extending from the rotation part to the entrance is sandwiched between the pair of rollers.

In an embodiment, the drum assembly may further include: an interlocking mechanism configured to rotate the rotation part and the pair of rollers in conjunction with each other.

According to an embodiment of the present disclosure, the translational movement of a surgical tool such as a catheter or a guide wire can be effectively implemented.

According to an embodiment of the present disclosure, the translational movement of the surgical tool can be accurately controlled by preventing buckling or bending in unintended portions when the surgical tool is translated.

According to an embodiment of the present disclosure, the translational and rotational movements of a surgical tool can be effectively implemented.

### BRIEF DESCRIPTION OF THE DRAWINGS

FIG. 1 schematically illustrates an example of insertion and rotation of a surgical tool in a vascular intervention.

FIG. 2 is a perspective view of a vascular intervention device according to an embodiment.

FIG. 3 is an exploded perspective view of a drum assembly according to an embodiment.

FIG. 4 is a plan view illustrating a rotation part and a pair of rollers of the drum assembly according to an embodiment.

FIG. 5 is a cross-sectional view of the drum assembly of FIG. 4 taken along line I-I′.

FIG. 6 is a cross-sectional view of the drum assembly of FIG. 4 taken along line II-II′.

FIG. 7 is a cross-sectional view of the drum assembly of FIG. 4 taken along line III-III′.

FIG. 8 is a plan view illustrating the interlocking mechanism of the drum assembly.

FIG. 9 is an exploded perspective view illustrating a supporter and a driving part according to an embodiment.

FIG. 10 is a top view of a vascular intervention device according to an embodiment.

FIG. 11 is a cross-sectional view of the vascular intervention device of FIG. 10 taken along line IV-IV′.

FIG. 12 is a cross-sectional view illustrating a state in which a translation driven shaft and a rotation driven shaft of a drum assembly according to an embodiment are not coupled to a translation driving shaft and a rotation driving shaft installed to the supporter.

FIG. 13 is a partial perspective view illustrating a structure that supports the drum assembly according to an embodiment on one side.

FIG. 14 is a partial perspective view illustrating a structure that supports the drum assembly according to an embodiment on the other side.

### DETAILED DESCRIPTION

Embodiments of the present disclosure are exemplified for describing the technical contents of the present disclosure. The scope of rights according to the present disclosure is not limited to the embodiments presented below or the specific descriptions of these embodiments.

All technical terms and scientific terms used in the present disclosure have meanings that are commonly understood by a person ordinarily skilled in the art to which the present disclosure belongs unless otherwise defined. All of the terms used in the present disclosure are selected for the purpose of describing the present disclosure more clearly, and are not selected to limit the scope of rights according to the present disclosure.

As used in the present disclosure, expressions such as “including,” “comprising,” “having,” and the like are to be understood as open-ended terms having the possibility of encompassing other embodiments, unless otherwise mentioned in the phrase or sentence including such expressions.

Singular expressions that are described in the present disclosure may encompass plural expressions unless otherwise stated, which also applies to the singular expressions recited in the claims.

As used in the present disclosure, expressions such as “first,” “second,” and the like are used to distinguish multiple elements from each other, and are not intended to limit an order or importance of the corresponding elements.

The dimensional and numerical values described in the present disclosure are not limited only to the dimensional and numerical values that are described herein. Unless specified otherwise, the dimensional and numerical values may be understood to mean the described values and equivalent ranges including the values. For example, a dimension “xx mm” described herein may be understood to include “about xx mm.”

Hereinafter, embodiments of the present disclosure will be described with reference to the accompanying drawings. In the accompanying drawings, the same or corresponding components are assigned the same reference numerals. In addition, in the description of the following embodiments, redundant descriptions of the same or corresponding components may be omitted. However, even if descriptions of components are omitted, it is not intended that such components are not included in any embodiment.

The embodiments of the present disclosure and the embodiments illustrated in the drawings relate to a vascular intervention device used to transport and rotate a surgical tool used in a vascular intervention and to insert the surgical tool into a target blood vessel. The vascular intervention device according to embodiments is used for a vascular intervention using a catheter, a guide wire, a micro-catheter, or a micro guide wire. In the present disclosure, a catheter, a guide wire, a micro-catheter, and a micro guide wire are referred to as surgical tools.

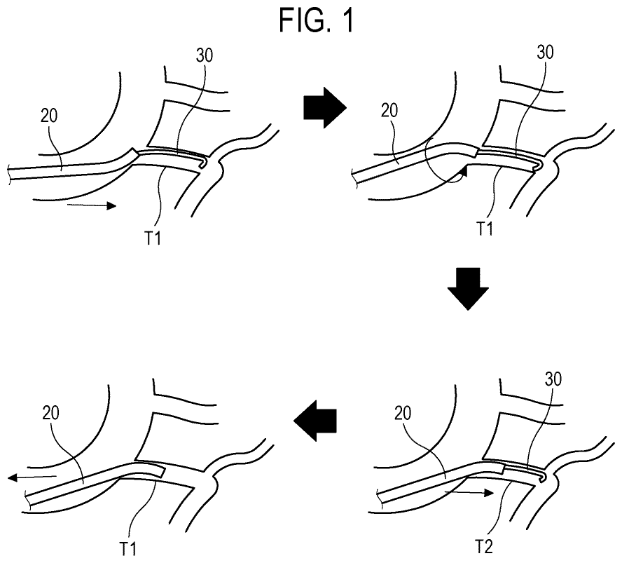

The catheter is a flexible tube that enters a target blood vessel. The guide wire is inserted into the catheter so as to guide the catheter to the target blood vessel. The micro-catheter is a flexible tube that is insertable into the catheter. The micro-catheter can enter a narrower target blood vessel that the catheter cannot enter, and is used to inject a drug into the narrower target blood vessel or to suction blood clots. The micro guide wire has a smaller thickness than the guide wire and is used to guide the micro-catheter into the narrower target blood vessel. The micro guide wire is inserted into the micro-catheter. FIG. 1 schematically illustrates an example of insertion and rotation of a surgical tool in a vascular intervention.

Referring to FIG. 1, an example of using a catheter and a guide wire by a vascular intervention device according to an embodiment to allow the catheter to reach a target vessel will be described. The vascular intervention device may transport a catheter 20 and a guide wire 30 in order to allow the catheter 20 and the guide wire 30 to enter the vicinity of a first target blood vessel T1. The vascular intervention device may transport and rotate the guide wire 30 in order to allow the guide wire 30 to enter the first target blood vessel T1. In addition, the vascular intervention device may rotate the catheter 20 along with the transport and rotation of the guide wire 30. When the guide wire 30 enters the first target blood vessel T1, the vascular intervention device may allow the catheter 20 to enter the first target blood vessel T1 along the guide wire 30. When the catheter 20 reaches the first target blood vessel T1, the guide wire 30 is removed from the catheter 20. The catheter 20 may be used to inject a drug into the first target blood vessel T1 or to suction blood clots within the first target blood vessel T1. Hereinafter, a vascular intervention device that implements the movement (transport and rotation) of a surgical tool will be described.

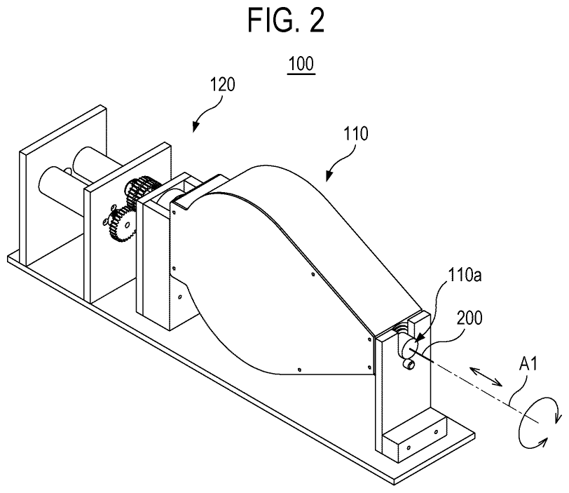

FIG. 2 is a perspective view of a vascular intervention device 100 according to an embodiment.

Referring to FIG. 2, in an embodiment, the vascular intervention device 100 may include a drum assembly 110 and a supporter 120. The vascular intervention device 100 may be configured to rotate the drum assembly 110 about a first axis A1. The “first axis A1” used in the present disclosure refers to a virtual axis as illustrated in FIG. 2. For example, the drum assembly 110 is rotatably installed on the supporter 120, and the vascular intervention device 100 may include a driving part that is able to rotate the drum assembly 110.

The drum assembly 110 is capable of accommodating a flexible wire-type or tube-type surgical tool 200 that is insertable into a blood vessel. The drum assembly 110 may be configured to pull the surgical tool 200 out of the drum assembly 110 or insert the surgical tool 200 into the drum assembly 110.

The drum assembly 110 includes an entrance 110a aligned on the first axis A1. The surgical tool 200 is able to enter and exit the drum assembly through the entrance 110a. The drum assembly 110 may be configured to allow the surgical tool 200 to enter and exit through the entrance 110a.

In an embodiment, the surgical tool 200 enters and exits the drum assembly 110 through the entrance 110a aligned on the first axis A1, and the drum assembly 110 is rotatable about the first axis A1. Accordingly, the vascular intervention device 100 is able to move the surgical tool 200 forward or rearward within the blood vessel and control the direction of the surgical tool 200.

In the present disclosure, the operation of the surgical tool 200 exiting the entrance 110a of the drum assembly 110 along the first axis A1 or entering the entrance 110a is referred to as the translational movement of the surgical tool 200. In addition, the movement of the surgical tool 200 exiting the drum assembly 110 is referred to as the advancement of the surgical tool 200, and the movement of the surgical tool 200 entering the drum assembly 110 is referred to as the retraction or retreat of the surgical tool 200. The length of the surgical tool 200 exiting the drum assembly 110 may be adjusted according to the translational movement of the surgical tool 200, and accordingly, the end of the surgical tool 200 may be moved to a desired point within the blood vessel.

In the present disclosure, the operation of the surgical tool 200 when the drum assembly 110 rotates about the first axis A1 is referred to as the rotation of the surgical tool 200. In the vicinity of the entrance 110a, the surgical tool 200 rotates about the first axis A1.

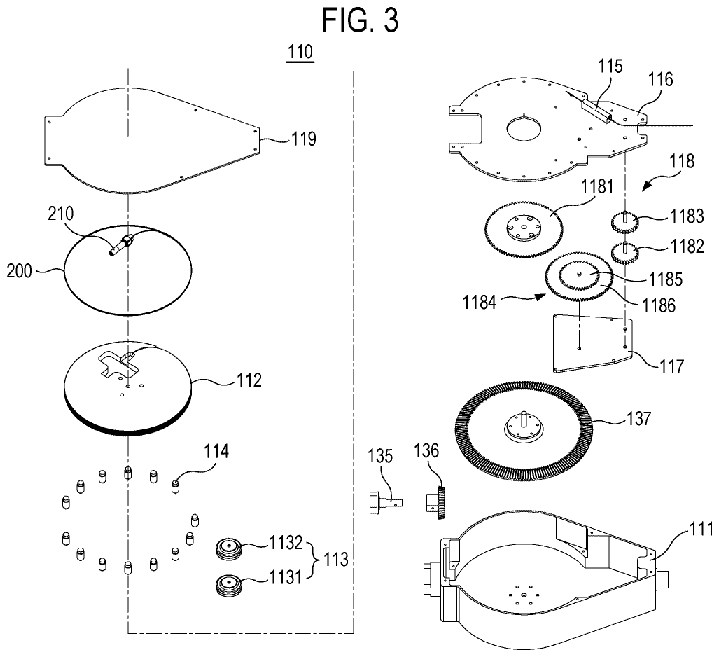

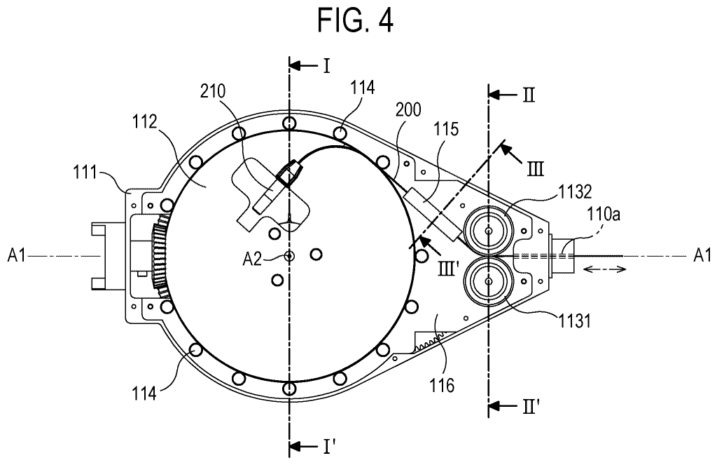

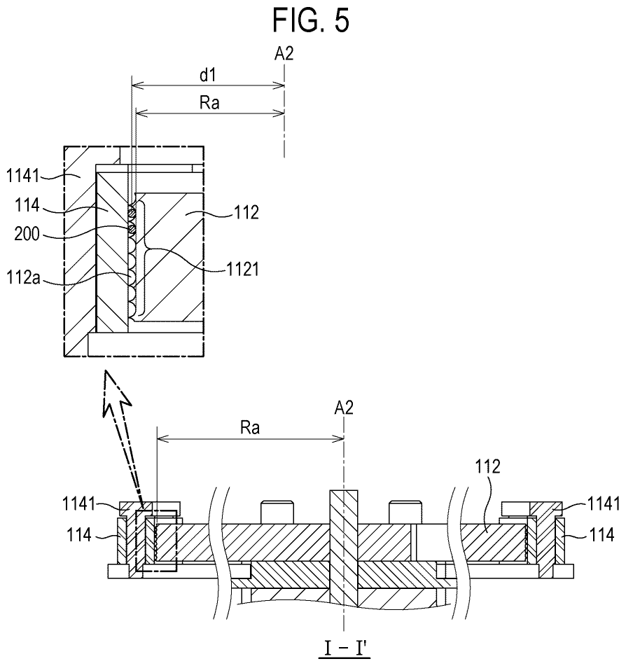

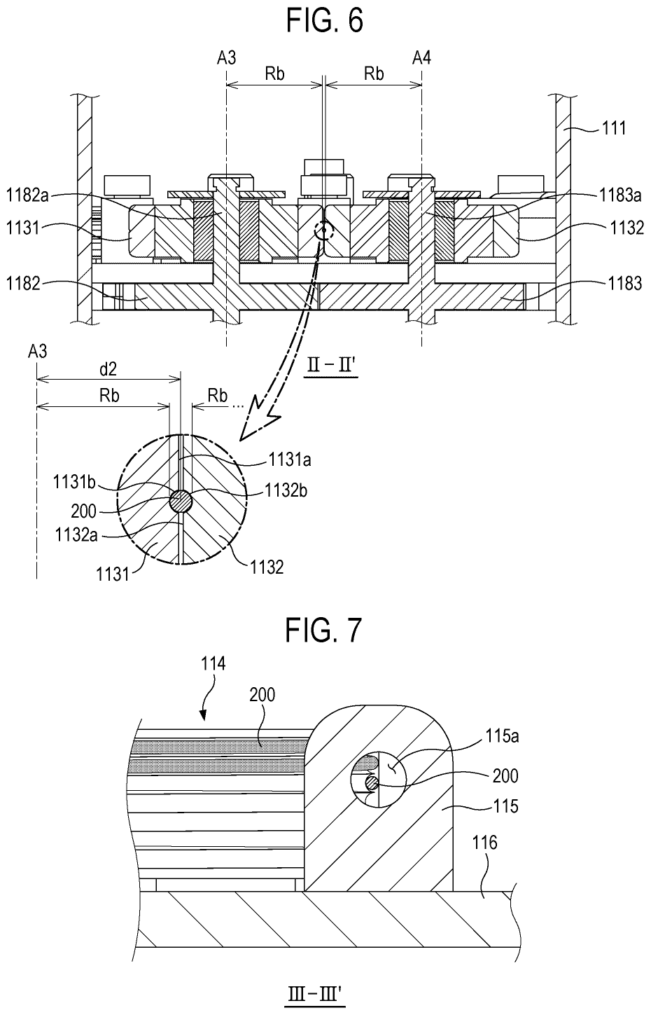

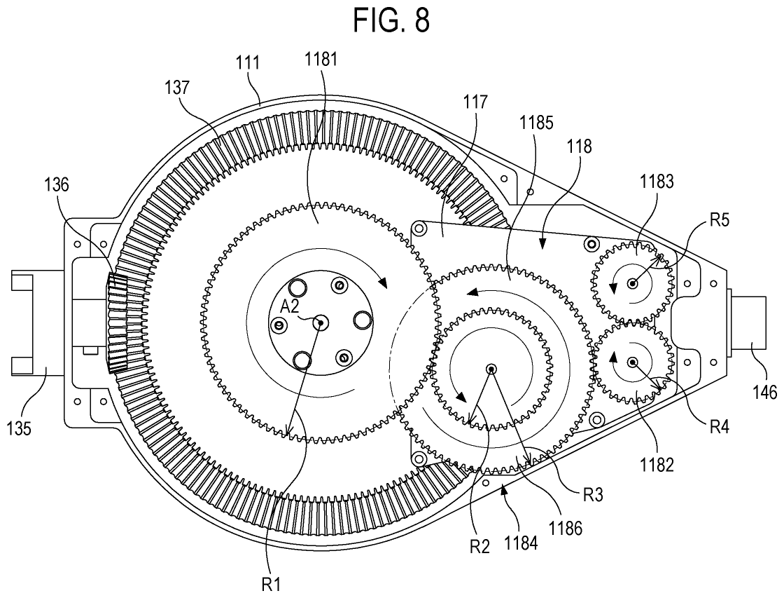

FIG. 3 is an exploded perspective view of the drum assembly 110 according to an embodiment. FIG. 4 is a plan view illustrating a rotation part and a pair of rollers of the drum assembly 110 according to an embodiment. FIG. 5 is a cross-sectional view of the drum assembly 110 of FIG. 4 taken along line I-I′. FIG. 6 is a cross-sectional view of the drum assembly 110 of FIG. 4 taken along line II-II′. FIG. 7 is a cross-sectional view of the drum assembly 110 of FIG. 4 taken along line III-III′. FIG. 8 is a plan view illustrating an interlocking mechanism of the drum assembly 110.

Referring to FIGS. 3 to 8, the drum assembly 110 may include a drum housing 111 and a rotation part 112 disposed inside the drum housing 111. A cover 119 may be coupled to the drum housing 111. The rotation part 112 may be configured to be rotatable. The rotation part 112 may be configured to be rotatable about a second axis A2. The “second axis A2” used in the present disclosure refers to a virtual axis as indicated in FIG. 4. For example, the rotation part 112 may be coupled to the drum housing 111 to be rotatable about the second axis A2.

The second axis A2 extends in a direction crossing the first axis A1. For example, the first axis A1 and the second axis A2 may be perpendicular or substantially perpendicular to each other. The second axis A2 may intersect the first axis A1. In the present disclosure, the second axis A2 is an axis fixed to the drum assembly 110 and rotates with the drum assembly 110 as the drum assembly 110 rotates.

The rotation part 112 is configured to wind the surgical tool 200 in a circumferential direction centered on the second axis A2. The rotation part 112 may include an outer peripheral surface, and the surgical tool 200 is wound around the outer peripheral surface. For example, the rotation part 112 is provided in a disk-like shape, and the surgical tool 200 may be wound around the outer peripheral surface of the disk.

The fixed end of the surgical tool 200 may be fixed to the rotation part 112 via a torque device. The free end of the surgical tool 200 may exit the drum assembly 110 through the entrance 110a and then advance or retreat according to the rotation of the rotation part 112.

When the rotation part 112 rotates about the second axis A2, the drum assembly 110 is able to guide the surgical tool 200 such that the surgical tool 200 advances or retreats through the entrance 110a. As the rotation part 112 rotates, the surgical tool 200 wound around the rotation part 112 may be released or further wound. Accordingly, the length of the surgical tool 200 pulled out of the drum assembly 110 is adjustable.

For example, referring to FIG. 4, when the rotation part 112 rotates counterclockwise about the second axis A2, the surgical tool 200 may be further wound around the outer peripheral surface of the rotation part 112, and the length of the surgical tool 200 exiting the drum assembly 110 to the outside may decrease. As another example, when the rotation part 112 rotates clockwise about the second axis A2, the surgical tool 200 may be released and the length of the surgical tool 200 exiting the drum assembly 110 may increase.

Referring to FIG. 5, a guide groove 1121 into which the surgical tool 200 can be seated may be formed on the outer peripheral surface of the rotation part 112. The guide groove 1121 may extend in a circumferential direction around the second axis A2. For example, the guide groove 1121 may extend in a spiral shape. In the embodiment illustrated in FIGS. 3 and 5, the guide groove 1121 extends as a circular groove of multiple turns along the outer peripheral surface of the rotation part. However, this is merely an example, and in other embodiments, the guide groove 1121 may be omitted or provided in other forms.

Referring to FIG. 4, the drum assembly 110 may include one or more guide rollers 114. The one or more guide rollers 114 may be disposed outside the outer peripheral surface of the rotation part 112. The guide rollers 114 may be arranged in the circumferential direction. Multiple guide rollers 114 may be arranged in the circumferential direction of the rotation part 112. In an embodiment, the guide rollers 114 may be fitted into the guide roller shaft 1141 installed on an intermediate plate 116.

The guide rollers 114 may be rotatable about an axis parallel to the second axis A2. When the rotation part 112 rotates, the surgical tool 200 wound around the rotation part 112 also rotates, and the guide rollers 114 may also rotate due to friction between the surgical tool 200 and the guide rollers 114. For example, when the rotation part 112 rotates clockwise about the second axis A2, the guide rollers 114 may rotate counterclockwise.

The guide rollers 114 allow the surgical tool 200 to be properly wound around the outer peripheral surface of the rotation part 112. For example, the surgical tool 200 may be wound along the guide groove 1121 formed on the outer peripheral surface of the rotation part 112, and the guide rollers 114 may help the surgical tool 200 maintain contact with the guide groove 1121.

The guide rollers 114 may be configured to be in contact with the surgical tool 200 wound around the rotation part 112. The surgical tool 200 may be sandwiched between the guide rollers 114 and the rotation part 112. Accordingly, the surgical tool 200 may be prevented from being separated from the rotation part 112.

The drum assembly 110 may include a pair of rollers 113 parallel to each other. The pair of rollers 113 may include a first roller 1131 and a second roller 1132 that are parallel to each other. The pair of rollers 113 may be configured to rotate in conjunction with the rotation part 112. For example, the angular velocities of the pair of rollers 113 relative to the angular velocity of the rotation part 112 may have a fixed value. This will be described in detail later with reference to FIG. 8.

The pair of rollers 113 may rotate about a rotation axis parallel to the second axis A2. The pair of rollers 113 may be coupled to the drum housing 111 to be rotatable about axes parallel to the second axis A2. For example, the first roller 1131 and the second roller 1132 rotate about a third axis A3 and a fourth axis A4, respectively, and both the third axis A3 and the fourth axis A4 may be parallel to the second axis.

The drum assembly 110 may be configured such that a portion of the surgical tool 200 extending from the rotation part 112 to the entrance 110a is sandwiched between the pair of rollers 113. Referring to FIG. 6, a gap exists between the outer peripheral surface 1131a of the first roller 1131 and the outer peripheral surface 1132a of the second roller 1132, and the surgical tool 200 may be sandwiched in the gap.

Grooves 1131b and 1132b may be formed on the outer peripheral surfaces of the pair of rollers 1131 and 1132, respectively. The grooves 1131b and 1132b may extend in the circumferential direction of the corresponding rollers 1131 and 1132. The first groove 1131b and the second groove 1132b may be formed at the same height. The surgical tool 200 may be sandwiched between the first groove 1131b and the second groove 1132b.

The pair of rollers 113 may be configured to rotate at the same angular speed but in opposite directions. For example, when the first roller 1131 rotates clockwise, the second roller 1132 may rotate counterclockwise. As the first roller 1131 and the second roller 1132 rotate in opposite directions, the first roller 1131 and the second roller 1132 may make the surgical tool 200 sandwiched therebetween move along the first axis A1. The interlocking between the pair of rollers 113 will be described in detail later with reference to FIG. 8.

The drum assembly 110 may include a surgical tool guide 115. For example, the surgical tool guide 115 may be disposed on the intermediate plate 116.

The surgical tool guide 115 may be configured to accommodate a portion of the surgical tool 200 extending between the rotation part 112 and the pair of rollers 113 and guide the movement of the surgical tool 200. Referring to FIG. 7, for example, the surgical tool guide 115 may include a hole 115a that accommodates the surgical tool 200. The surgical tool guide 115 helps the portion of the surgical tool 200 that extends from the rotation part 112 toward the pair of rollers 113 to fit well between the pair of rollers 113.

The drum assembly 110 may include an interlocking mechanism 118 configured to rotate the rotation part 112 and the pair of rollers 113 in conjunction with each other.

The interlocking mechanism 118 may be configured such that the length of the surgical tool 200 unwound from or wound onto the rotation part 112 per unit time as the rotation part 112 rotates matches or substantially matches the distance the pair of rollers 113 are in contact with the surgical tool 200 per unit time. Meanwhile, for convenience of description in the present disclosure, the condition where “the length of the surgical tool 200 unwound from or wound onto the rotation part 112 per unit time as the rotation part 112 rotates matches the distance the pair of rollers are in contact with the surgical tool 200 per unit time” is referred to as a “linear velocity condition.” That is, the interlocking mechanism 118 may be configured to satisfy the linear velocity condition.

In an embodiment, the rotation part 112 may include an outer peripheral surface configured to wind the surgical tool 200 therearound (e.g., the guide groove 1121 in FIG. 5), and the pair of rollers 113 may each have an outer peripheral surface configured to be in contact with the surgical tool 200 (e.g., the first groove 1131b and the second groove 1132b in FIG. 6). In an embodiment, the interlocking mechanism 118 may be configured such that, when the rotation part 112 and the pair of rollers 113 rotate, the outer peripheral surfaces of the rotation part 112 and the pair of rollers 113 have the same outer peripheral linear velocity. In the present disclosure, unless otherwise stated, the outer peripheral linear velocity refers to the linear velocity of the portion of an outer peripheral surface that is in contact with the surgical tool 200.

Referring to FIGS. 5 and 6, in an embodiment, if the product of the distance d1 from the second axis A2 to the center of the surgical tool 200 and the angular velocity of the rotation part 112 matches the product of the distance d2 from the third axis A3 (or the fourth axis A4) to the center of the surgical tool 200 sandwiched between the pair of rollers 113 and the angular velocity of the pair of rollers 113, the linear velocity condition can be satisfied. In an embodiment, since the diameter of the surgical tool 200 is relatively smaller compared to the diameter of the rotation part 112 or the pair of rollers 113, if the product of the distance Ra from the second axis A2 to the guide groove 1121 and the angular velocity of the rotation part 112 equals the product of the distance Rb from the third axis A3 (or the fourth axis A4) to the first groove 1131b of the first roller 1131 (or the second groove 1132b of the second roller 1132) (or the second groove 1132b) and the angular velocity of the pair of rollers 113, the linear velocity condition can be substantially satisfied.

When the linear velocity condition is satisfied, the tension of the surgical tool 200 extending between the rotation part 112 and the pair of rollers 113 can be maintained constant, thereby allowing the surgical tool 200 to smoothly translate. Since the tension is maintained constant, the surgical tool 200 can be prevented from buckling or bending between the rotation part 112 and the pair of rollers 113, thereby enabling precise control of the translational movement of the surgical tool 200. When it is described that the linear velocity condition is substantially satisfied, it means that the same technical effect as when the linear speed condition is strictly satisfied is achieved.

In an embodiment, the interlocking mechanism 118 may be configured such that the outer peripheral linear velocity of the rotation part 112 and the outer peripheral linear velocity of the pair of rollers 113 match or substantially match each other. Here, the outer peripheral linear velocity of the rotation part 112 refers to the linear velocity of the portion of the rotation part 112 that is in contact with the surgical tool 200. In addition, the outer peripheral linear velocity of the pair of rollers 113 refers to the linear velocity of the portions of the pair of rollers 113 that are in contact with the surgical tool 200. For example, referring to FIG. 5, the surgical tool 200 is in contact with the groove of the rotation part 112, and the outer peripheral linear velocity of the rotation part 112 may be the linear velocity of the guide groove 1121 having the first radius Ra. As another example, referring to FIG. 6, the surgical tool 200 is in contact with the first groove 1131b and the second groove 1132b, and the outer peripheral linear velocity of the pair of rollers 113 may be the linear velocity of the first groove 1131b (or the second groove 1132b) having the second radius Rb.

In an embodiment, the interlocking mechanism 118 may be configured to rotate the pair of rollers 113 in opposite directions. For example, when the first roller 1131 rotates clockwise, the second roller 1132 may rotate counterclockwise by the interlocking mechanism 118. Referring to FIGS. 6 and 8, a first driven gear 1182 coupled to the first roller 1131 and a second driven gear 1183 coupled to the second roller 1132 may be engaged with each other, and accordingly, the first roller 1131 and the second roller 1132 may rotate in opposite directions.

In an embodiment, the interlocking mechanism 118 may rotate the pair of rollers 113 in opposite directions and at the same linear velocity. For example, the pitch circle of the first driven gear 1182 and the pitch circle of the second driven gear 1183 have the same radii (R4=R5), and the first roller 1131 and the second roller 1132 may have the same outer peripheral radius (Rb). Here, the outer peripheral radius refers to the radius of the portions of the outer peripheral surfaces of the rollers 1131 and 1132 that are in contact with the surgical tool 200 (e.g., the radius of the first groove 1131b or the second groove 1132b).

In an embodiment, the interlocking mechanism 118 may include a plurality of gears that rotate in conjunction with each other. For example, the interlocking mechanism 118 may include a rotation part gear 1181 coupled to the rotation part 112, the first driven gear 1182 coupled to one of the pair of rollers 113, and the second driven gear 1183 coupled to another one of the pair of rollers 113 and engaged with the first driven gear 1182.

In an embodiment, the rotation part gear 1181 and the first driven gear 1182 may be interlocked through an intermediate element 1184. The intermediate element 1184 may include a first intermediate gear 1185 engaged with the rotation part gear 1181 and a second intermediate gear 1186 engaged with the first driven gear 1182. The first intermediate gear 1185 and the second intermediate gear 1186 may have pitch circles that are concentric with each other. The first intermediate gear 1185 and the second intermediate gear 1186 form an integrated part and may rotate together at the same angular speed. For example, the second intermediate gear 1186 may be fixedly coupled to the first intermediate gear 1185.

Referring to FIG. 3, in an embodiment, the interlocking mechanism 118 may be disposed between the intermediate plate 116 and an auxiliary plate 117. The intermediate plate 116 and the auxiliary plate 117 may be configured to support the rotation of the first driven gear 1182, the second driven gear 1183, and the intermediate element 1184. For example, referring to FIG. 6, one side of the shafts 1182a and 1183b of the first and second driven gears 1182 and 1183 is fitted into the intermediate plate 116, and the other side is fitted into the auxiliary plate 117.

Referring to FIG. 8, the pitch circle of the rotation part gear 1181 is in contact with the pitch circle of the first intermediate gear 1185, and the pitch circle of the second intermediate gear 1186 is in contact with the pitch circle of the first driven gear 1182. The pitch circle of the first driven gear 1182 and the pitch circle of the second driven gear 1183 have the same radius and are in contact with each other.

The plurality of gears may be configured such that the outer peripheral linear velocity of the rotation part 112 matches the outer peripheral linear velocity of the pair of rollers 113. When the pitch radius of the rotation part gear 1181 is R1, the pitch radius of the first intermediate gear is R2, the pitch radius of the second intermediate gear is R3, the pitch radius of the first roller is R4, and the outer peripheral radius of the rotation part 112 is Ra and the outer peripheral radius of the first roller 1131 is Rb, the following Equation 1 may be satisfied.

R4/R1=Rb/Ra*R3/R2 [Equation 1]

Meanwhile, in another embodiment (not illustrated), the interlocking mechanism 118 may be configured such that only the first roller 1131 of the pair of rollers 113 operates in conjunction with the rotation part 112. For example, the second driven gear 1183 may be omitted from the interlocking mechanism 118. Accordingly, the second roller 1132 is freely rotatable. In this case, the first roller 1131 may move the surgical tool 200 while operating in conjunction with the rotation part 112, and the second roller 1132 may rotate in the opposite direction to the first roller 1131 due to friction between the surgical tool 200 and the second roller 1132. In this embodiment, the first roller 1131 and the second roller 1132 may have different outer peripheral radii.

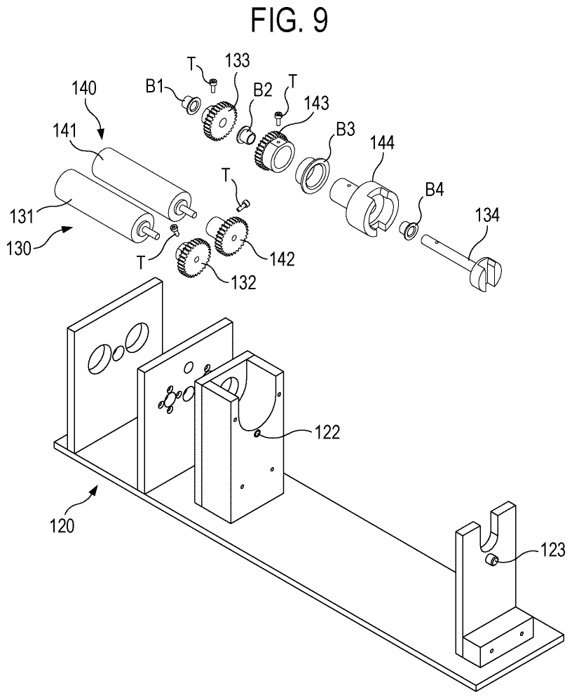

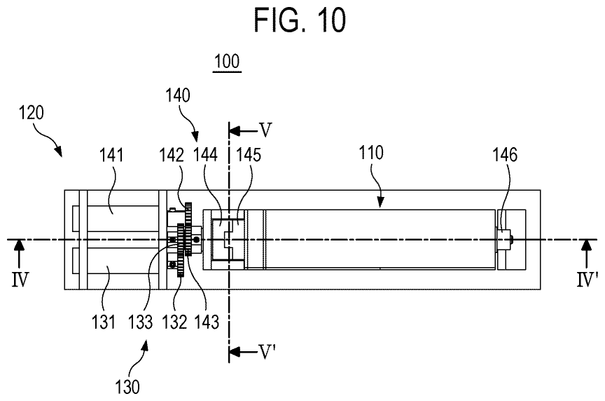

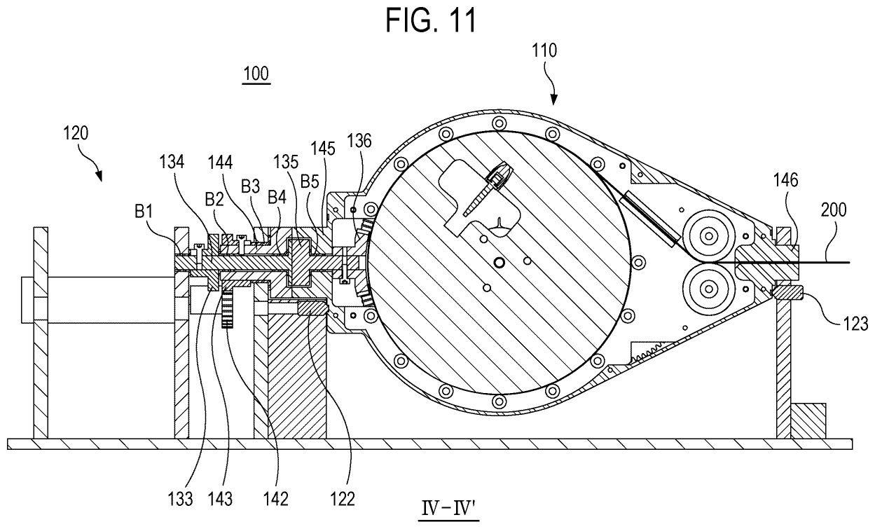

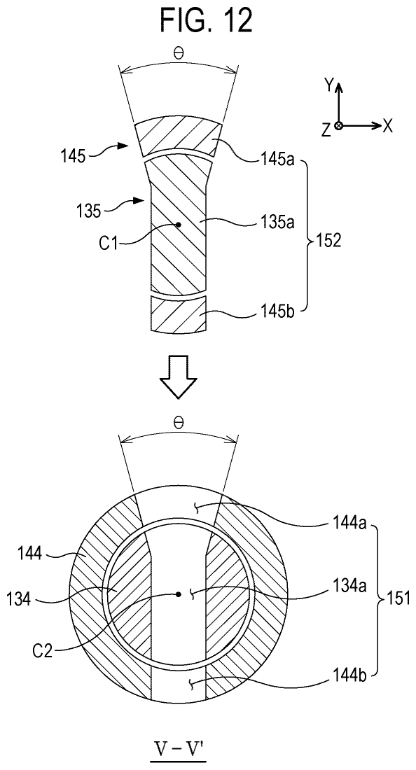

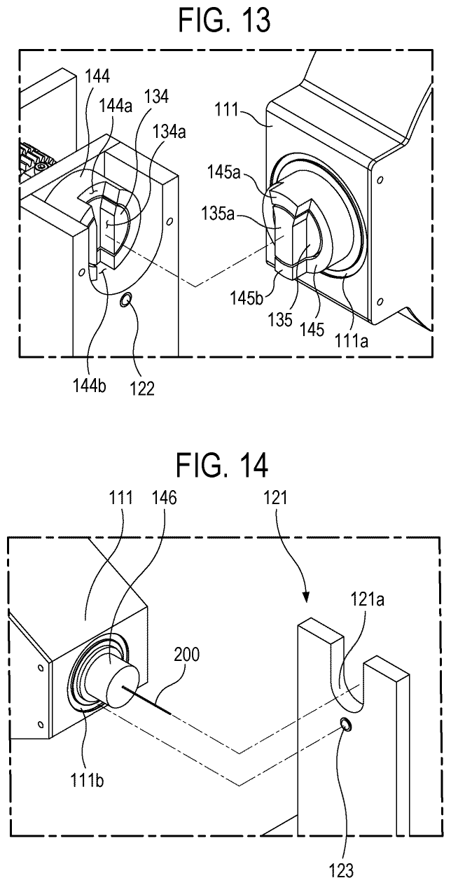

FIG. 9 is an exploded perspective view illustrating the supporter 120 and the driving part according to an embodiment. FIG. 10 is a top view of the vascular intervention device 100 according to an embodiment. FIG. 11 is a cross-sectional view of the vascular intervention device 100 of FIG. 10 taken along line IV-IV′. FIG. 12 is a cross-sectional view illustrating a state in which the translation driven shaft 135 and a rotation driven shaft 145 of the drum assembly 110 according to an embodiment are not coupled to a translation driving shaft 134 and a rotation driving shaft 144 installed on the supporter 120. FIG. 12 is a cross-sectional view of the vascular intervention device 100 of FIG. 10 taken along line V-V′. FIG. 13 is a partial perspective view illustrating a structure that supports the drum assembly 110 according to an embodiment on one side. FIG. 14 is a partial perspective view illustrating a structure that supports the drum assembly 110 according to an embodiment on the other side.

Referring to FIGS. 9 to 11, the vascular intervention device 100 includes a rotation part 112 and a translation driver 130 configured to rotate a pair of rollers. The translation driver 130 may include a translation driven shaft 135 that is installed to the drum assembly 110 and rotates in mechanical conjunction with the rotation part 112. The translation driver 130 may include a translation driving shaft 134 installed on the supporter 120 and configured to transmit power to the translation driven shaft 135. For example, the translation driving shaft 134 and the translation driven shaft 135 may be mechanically engaged with each other and rotate together.

Referring to FIGS. 8 to 11, the translation driver 130 may include a first bevel gear 136 coupled to the first translation driven shaft 135 and a second bevel gear 137 engaged with the first bevel gear 136 and coupled to the rotation part 112.

The translation driver 130 may include a first motor 131, a first spur gear 132, and a second spur gear 133. The first spur gear 132 may be coupled to the output shaft of the first motor 131, and the second spur gear 133 may be engaged with the first spur gear 132 and coupled to the translation driving shaft 134. When the first motor 131 is driven, power may be sequentially transmitted through the first spur gear 132, the second spur gear 133, the translation driving shaft 134, the translation driven shaft 135, the first bevel gear 136, and the second bevel gear 137 to rotate the rotation part 112. In the present disclosure, the combination of gears that transmit power from the first motor 131 to the rotation part 112 is only an example, and power may be transmitted through various gear combinations in other embodiments. For example, the translation driver 130 may include a power transmission element such as a driving chain or a driving belt.

Referring to FIGS. 9 to 11, the vascular intervention device 100 includes a rotation driver 140 configured to rotate the drum assembly 110 about the first axis A1 with respect to the supporter 120. In an embodiment, the rotation driver 140 may include a rotation driven shaft 145 that is fixedly coupled to the drum assembly 110. For example, the rotation driven shaft 15 may be defined by the drum housing 111.

The rotation driver 140 may include a rotation driving shaft 144 installed on the supporter 120 and configured to transmit power to the rotation driven shaft 145. For example, the rotation driving shaft 144 and the rotation driven shaft 145 may be mechanically engaged with each other and rotate together.

Referring to FIG. 11, the rotation driven shaft 145 may include a hollow configured to accommodate the translation driven shaft 135. Both the rotation driven shaft 145 and the translation driven shaft 135 may rotate about the first axis A1, and the hollow may extend along the first axis A1.

The rotation driving shaft 144 may include a hollow configured to accommodate the translation driving shaft 134. Both the rotation driving shaft 144 and the translation driving shaft 134 rotate about the first axis A1, and the hollow portion may extend along the first axis A1.

The rotation driver 140 may include a second motor 141, a third spur gear 142, and a fourth spur gear 143. The third spur gear 142 may be coupled to the output shaft of the second motor 141, and the fourth spur gear 143 may be engaged with the third spur gear 142 and coupled to the rotation driving shaft 144. When the second motor 141 is driven, power may be sequentially transmitted through the third spur gear 142, the fourth spur gear 143, the rotation driving shaft 144, and the rotation driven shaft 145 to rotate the drum assembly 110. In the present disclosure, the combination of gears that transmit power from the second motor 141 to the rotation driven shaft 145 is only an example, and power may be transmitted through various gear combinations in other embodiments. For example, the rotation driver 140 may include a power transmission element such as a driving chain or a driving belt.

Referring to FIG. 9, gears may be fixedly coupled to corresponding shafts via fastening members T. For example, the first spur gear 132 may be fixedly coupled to the output shaft of the first motor 131 via a fastening member T. As another example, the fourth spur gear 143 may be fixed to the translation driving shaft 144 via a fastening member T.

Referring to FIGS. 9 and 10, bearings B1, B2, B3, B4, and B5 may be disposed between elements that rotate with each other. For example, the bearings B2 and B4 may be disposed between the translation driving shaft 134 and the rotation driving shaft 144 to reduce friction due to relative movement between the two shafts. As another example, one side of the translation driving shaft 134 is supported by the supporter 120, and the bearing B1 may be disposed between the supporter 120 and the translation driving shaft 134. As another example, the bearing B5 may be disposed between the translation driven shaft 135 and the rotation driven shaft 145. As another example, the bearing B3 may be disposed between the rotation driving shaft 144 and the supporter 120.

The drum assembly 110 may be detachably installed to the supporter 120. After the surgical procedure, the drum assembly 110 including the contaminated surgical tool 200 may be removed from the supporter 120, and a new drum assembly may be installed to the supporter 120.

One of the translation driven shaft 135 and the translation driving shaft 134 may include a translation protrusion 135a protruding axially, and the other of the translation driven shaft 135 and the translation driving shaft 134 may include a translation groove 134a configured to be engaged with the translation protrusion 135a.

Referring to FIGS. 12 and 13, the translation protrusion 135a may extend in a first direction perpendicular to the central axis C1 of the translation driven shaft 135. For example, when the rotation axis of the translation driven shaft 135 is parallel to the Z-axis, the translation protrusion 135b may extend in the Y-axis direction.

One end of the translation protrusion 135a in the first direction may have a circumferential width that increases in the radial direction. One end of the translation protrusion 135a in the first direction may have a larger width in a direction away from the center. Here, the end of the translation protrusion 135a includes a portion close to the distal end of the translation protrusion 135a. The circumferential width of the translation groove 134a may also be provided in a radially increasing form. For example, referring to FIG. 11, the upper portion of the translation protrusion 135a is partially defined by inclined surfaces forming an angle θ relative to each other, and the upper portion of the translation groove 134a may be partially defined by inclined surfaces forming the angle θ. For example, the translation protrusion 135a may have a “Y”-shaped cross-section when viewed axially (i.e., in the Z-axis direction).

The translation protrusion 135a is inserted into the translation groove 134a in the direction of the arrow until the central axis C1 of the translation driven shaft 135 and the central axis C2 of the translation driving shaft 134 coincide with each other. When the central axes C1 and C2 of the translation driven shaft 135 and the translation driving shaft 134 coincide with each other, the translation protrusion 135a and the translation groove 134a are engaged with each other, and the translation protrusion 135a no longer moves in the direction of the arrow.

One of the rotation driven shaft 145 and the rotation driving shaft 144 may include a rotation protrusion 145a protruding axially, and the other of the rotation driven shaft 145 and the rotation driving shaft 144 may include a rotation groove 144a configured to be engaged with the rotation protrusion 145a. For example, the rotation protrusion 145a may be formed on the rotation driven shaft 145, and the rotation groove 144a may be formed on the rotation driving shaft 144.

The rotation protrusion 145a may be provided in a shape in which the circumferential width thereof increases in the radial direction. The width of the rotation protrusion 145a may increase in a direction away from the rotation axis. The circumferential width of the rotation groove 144a may be provided in a radially increasing form. For example, the rotation protrusion 145a may be partially defined as inclined surfaces forming an angle θ relative to each other, and the rotation groove 144a may be partially defined as inclined surfaces forming an angle θ.

The rotation protrusion 145a is inserted into the rotation groove 144a in the direction of the arrow until the central axis C1 of the rotation driven shaft 145 and the central axis C2 of the rotation driving shaft 144 coincide with each other. When the central axes C1 and C2 of the rotation driven shaft 145 and the rotation driving shaft 144 coincide with each other, the rotation protrusion 145a and the rotation groove 144a are engaged with each other, and the rotation protrusion 145a no longer moves in the direction of the arrow.

One of the rotation driven shaft 145 and the rotation driving shaft 144 may include a third protrusion 145b protruding in the axial direction, and the other of the rotation driven shaft 145 and the rotation driving shaft 144 may include a third groove 144b configured to be engaged with the third protrusion 145b. For example, the third protrusion 145b may be formed on the rotation driven shaft 145, and the third groove 144b may be formed on the rotation driving shaft 144. The rotation protrusion 145a and the third protrusion 145b may be disposed in opposite directions with respect to the central axis C2 and may have different shapes. For example, the third protrusion 145b may have the same width in the circumferential direction, unlike the rotation protrusion 145a.

Since the translation driven shaft 135 and the rotation driven shaft 145 rotate independently of each other, the translation protrusion 135a and the rotation protrusion 145a are not always aligned as illustrated in FIG. 12. Likewise, since the translation driving shaft 134 and the rotation driving shaft 144 rotate independently of each other, the translation groove 134a and the rotation groove 144a are not always aligned as illustrated in FIG. 12. When mounting the drum assembly 110 on the supporter 120, a user may mutually align the translation protrusion 135a and the rotation protrusion 145a in the form illustrated in the upper portion of FIG. 12, and may mutually align the translation groove 134a and the rotation groove 144a in the form illustrated in the lower portion of FIG. 12. The translation protrusion 135a and the rotation protrusion 145a may form an alignment protrusion 152 by being mutually aligned, and the translation groove 134a and the rotation groove 144a may form an alignment groove 151 by being mutually aligned. The alignment protrusion 152 may further include a third protrusion 145b. The alignment groove 151 may further include a third groove 144b.

The alignment protrusion 152 and the alignment groove 151 may be configured to be engaged with each other. The alignment protrusion 152 and the alignment groove 151 may be configured such that the alignment protrusion 152 is inserted into the alignment groove 151 in a direction perpendicular to the first axis A1. For example, referring to FIG. 12, the alignment protrusion 152 may be fitted into the alignment groove 151 in the direction of the arrow perpendicular to the first axis A1.

The drum assembly 110 may include an annular groove that is depressed in one direction of the first axis A1 and extends in a circumferential direction about the first axis A1. The supporter 120 may include a pin member that protrudes in one direction of the first axis A1 and is inserted into the annular groove.

Referring to FIG. 13, the drum assembly 110 may include a first annular groove 111a, and the supporter 120 may include a first pin member 122 disposed at a position corresponding to the first annular groove 111a. As another example, referring to FIG. 14, the drum assembly 110 may include a second annular groove 111b, and the supporter 120 may include a second pin member 123 disposed at a position corresponding to the second annular groove 111b.

The annular groove may be defined by the drum housing 111. For example, the drum housing 111 may be injection-molded in a shape including the annular grooves 111a and 111b.

When the translation driven shaft 135 and the rotation driven shaft 145 are fitted into the translation driving shaft 134 and the rotation driving shaft 144, respectively, the first pin member 122 is accommodated in the first annular groove 111a. When the support shaft 146 is seated on a shaft support portion 121a of the frame 121, the second pin member 123 is accommodated in the second annular groove 111b.

The interaction of the pin members 122 and 123 and the annular grooves 111a and 111b prevents the drum assembly 110 from being separated from the supporter 120 during rotation.

In the foregoing, the technical idea of the present disclosure has been described with reference to some embodiments and examples illustrated in the accompanying drawings. However, it is to be understood that various substitutions, modifications, and alterations may be made without departing from the technical idea and scope of the present disclosure that can be understood by a person ordinarily skilled in the technical field to which the present disclosure pertains. In addition, such substitutions, modifications, and alterations are to be considered as falling within the scope of the appended claims.

What is claimed is:

1. A vascular intervention device comprising:
a supporter; and
a drum assembly coupled to the supporter to be rotatable about a first axis, the drum assembly being configured to accommodate a flexible wire-type or tube-type surgical tool configured to be insertable into a blood vessel, and to allow the surgical tool to enter and exit through an entrance aligned on the first axis,
wherein the drum assembly includes:
a rotation part configured to be rotatable about a second axis and configured such that the surgical tool is wound around the rotation part in a circumferential direction about the second axis; and
a pair of rollers configured to interlock with the rotation part and having a rotation axis parallel to the second axis, and
wherein the drum assembly is configured such that, when the rotation part rotates about the second axis, the surgical tool is guided to be pulled out from or inserted into the drum assembly through the entrance, and such that a portion of the surgical tool extending from the rotation part to the entrance is sandwiched between the pair of rollers.

2. The vascular intervention device of claim 1, wherein the drum assembly further includes an interlocking mechanism configured to rotate the rotation part and the pair of rollers in conjunction with each other.

3. The vascular intervention device of claim 2, wherein the rotation part includes an outer peripheral surface, and the surgical tool is wound around the outer peripheral surface,
wherein the pair of rollers each includes an outer peripheral surface configured to come into contact with the surgical tool, and
wherein the interlocking mechanism is configured such that, when the rotation part and the pair of rollers rotate, the outer peripheral surface of the rotation part and the outer peripheral surfaces of the pair of rollers have a same outer peripheral linear velocity.

4. The vascular intervention device of claim 2, wherein the interlocking mechanism is configured to rotate the pair of rollers in opposite directions from each other.

5. The vascular intervention device of claim 3, wherein the interlocking mechanism includes a plurality of gears that rotate in conjunction with each other, and
wherein the plurality of gears include:
a rotation part gear coupled to the rotation part;
a first driven gear coupled to one of the pair of rollers; and
a second driven gear coupled to the other one of the pair of rollers and engaged with the first driven gear.

6. The vascular intervention device of claim 5, wherein the plurality of gears include:
a first intermediate gear engaged with the rotation part gear; and
a second intermediate gear fixedly coupled to the first intermediate gear and having a pitch circle concentric with a pitch circle of the first intermediate gear, and
wherein the first driven gear is engaged with the second intermediate gear.

7. The vascular intervention device of claim 1, further comprising:
a translation driver configured to rotate the rotation part and the pair of rollers,
wherein the translation driver includes:
a translation driven shaft installed to the drum assembly and configured to rotate in mechanical conjunction with the rotation part; and
a translation driving shaft installed to the supporter and configured to transmit power to the translation driven shaft.

8. The vascular intervention device of claim 7, wherein one of the translation driven shaft and the translation driving shaft includes a translation protrusion that axially protrudes, and the other one of the translation driven shaft and the translation driving shaft includes a translation groove configured to be engaged with the translation protrusion.

9. The vascular intervention device of claim 8, wherein the translation protrusion extends in a first direction perpendicular to a rotation axis of the translation driven shaft.

10. The vascular intervention device of claim 9, wherein one end of the translation protrusion in the first direction is provided in a shape in which a circumferential width thereof increases in a radial direction.

11. The vascular intervention device of claim 7, further comprising:
a rotation driver configured to rotate the drum assembly about the first axis with respect to the supporter,
wherein the rotation driver includes:
a rotation driven shaft fixedly coupled to the drum assembly; and
a rotation driving shaft installed to the supporter and configured to transmit power to the rotation driven shaft,
wherein the rotation driven shaft includes a hollow configured to accommodate the translation driven shaft, and
wherein the rotation driving shaft includes a hollow configured to accommodate the translation driving shaft.

12. The vascular intervention device of claim 11, wherein one of the rotation driven shaft and the rotation driving shaft includes a rotation protrusion that axially protrudes, and the other one of the rotation driven shaft and the rotation driving shaft includes a rotation groove configured to be engaged with the rotation protrusion.

13. The vascular intervention device of claim 12, wherein the rotation protrusion is provided in a shape in which a circumferential width thereof increases in a radial direction.

14. The vascular intervention device of claim 12, wherein one of the translation driven shaft and the translation driving shaft includes a translation protrusion that axially protrudes, and the other one of the translation driven shaft and the translation driving shaft includes a translation groove configured to be engaged with the translation protrusion, and
wherein the translation groove and the rotation groove are configured to form an alignment groove by being mutually aligned,
wherein the translation protrusion and the rotation protrusion are configured to form an alignment protrusion by being mutually aligned, and
wherein the alignment protrusion and the alignment groove are configured to be engaged with each other.

15. The vascular intervention device of claim 14, wherein the alignment protrusion and the alignment groove are configured such that the alignment protrusion is inserted into the alignment groove in a direction perpendicular to the first axis.

16. The vascular intervention device of claim 1, wherein the drum assembly includes at least one guide roller configured to be rotatable about a rotation axis parallel to the second axis, arranged in a circumferential direction of the rotation part, and configured to come into contact the surgical tool wound around the rotation part.

17. The vascular intervention device of claim 1, further comprising:
a surgical tool guide configured to accommodate a portion of the surgical tool extending from the rotation part to a space between the pair of rollers and guide movement of the surgical tool.

18. The vascular intervention device of claim 1, wherein the drum assembly includes an annular groove that is depressed in a direction of the first axis and extends in a circumferential direction around the first axis, and
wherein the supporter includes a pin member that protrudes in the direction of the first axis and is inserted into the annular groove.

19. A drum assembly for a vascular intervention device, the drum assembly comprising:
a drum housing configured to accommodate a flexible wire-type or tube-type surgical tool that is insertable into a blood vessel;
an entrance aligned on a first axis to allow the surgical tool to enter and exit;
a rotation part configured to be rotatable about a second axis with respect to the drum housing and configured such that the surgical tool is wound around the rotation part in a circumferential direction about the second axis; and
a pair of rollers configured to interlock with the rotation part and having a rotation axis parallel to the second axis, and
wherein the drum assembly is configured such that, when the rotation part rotates about the second axis, the surgical tool is guided to be pulled out from or inserted into the drum assembly through the entrance, and such that a portion of the surgical tool extending from the rotation part to the entrance is sandwiched between the pair of rollers.

20. The drum assembly of claim 19, further comprising:
an interlocking mechanism configured to rotate the rotation part and the pair of rollers in conjunction with each other.
